(19) United States
(12) Patent Application Publication
(10) Pub. No.: US 2025/0102165 A1
(43) Pub. Date: Mar. 27, 2025

(54) AIR CONDITIONING SYSTEM AND AIR CONDITIONING METHOD

(71) Applicants: The University of Tokyo, Tokyo (JP); CHIYODA CORPORATION, Yokohama-shi (JP); SHIMIZU CORPORATION, Tokyo (JP)

(72) Inventors: Hiroji EBE, Tokyo (JP); Masakazu SUGIYAMA, Tokyo (JP); Jun MATSUMOTO, Yokohama-shi (JP); Dai TAKEDA, Yokohama-shi (JP); Ittetsu OBA, Yokohama-shi (JP); Shinya HASHIMOTO, Yokohama-shi (JP); Yukinori FUSE, Tokyo (JP); Mitsuhiro SUMIKURA, Tokyo (JP); Yoshito ARAI, Tokyo (JP)

(73) Assignees: The University of Tokyo, Tokyo (JP); CHIYODA CORPORATION, Yokohama-shi (JP); SHIMIZU CORPORATION, Tokyo (JP)

(21) Appl. No.: 18/858,989

(22) PCT Filed: Mar. 7, 2023
(86) PCT No.: PCT/JP2023/008547

§ 371 Date: Oct. 22, 2024

(30) Foreign Application Priority Data
Apr. 27, 2022 (JP) 2022-073568

(51) Int. Cl. F24F 8/60; C25B 1/02; C25B 3/01; C25B 3/26; C25B 15/02; C25B 15/08; F24F 8/10

(52) U.S. Cl. CPC F24F 8/60; C25B 1/02; C25B 3/01; C25B 3/26; C25B 15/02; C25B 15/08; F24F 8/10

(57) ABSTRACT

An air conditioning system (1) includes a carbon dioxide separation device (20) configured to separate some or all of carbon dioxide from air that contains carbon dioxide, an electrolytic reduction device (30) configured to generate a hydrocarbon and oxygen using the separated carbon dioxide as a raw material, and an oxygen supply amount control device (40) configured to supply some or all of the oxygen generated by the electrolytic reduction device (30) to a space to be processed (10).

### TECHNICAL FIELD

The present invention relates to an air conditioning system and an air conditioning method. Priority is claimed on Japanese Patent Application No. 2022-073568, filed Apr. 27, 2022, the content of which is incorporated herein by reference.

### BACKGROUND ART

The Building Environmental Sanitation Management Standards stipulate that the carbon dioxide content in a room equipped with an air conditioning facility must be 1,000 ppm (on a volume basis; the same applies hereinafter in this specification) or less. Thus, there is a demand for a technology for removing carbon dioxide from indoor air in buildings equipped with air conditioning facilities.

For example, Patent Document 1 proposes an air conditioning system that includes a rotor divided into a processing zone in which air to be processed, containing carbon dioxide, is absorbed by an amine-carrying solid absorbent agent, and a regeneration zone in which the carbon dioxide absorbed by the absorbent agent is desorbed into regeneration air, and that is configured so that an enthalpy difference between the air to be processed supplied to the processing zone and the regeneration air supplied to the regeneration zone is within a specific range. According to the invention of Patent Document 1, carbon dioxide in the indoor air is removed to improve quality of the air.

### CITATION LIST

### Patent Document

[Patent Document 1]

Japanese Unexamined Patent Application, First Publication No. 2017-75715

### SUMMARY OF INVENTION

### Technical Problem

Incidentally, carbon dioxide can be used to generate valuable materials if it is recovered by an appropriate method. In the technology of Patent Document 1, the removed carbon dioxide is discharged outdoors, and effective utilization of carbon dioxide is not taken into consideration. In addition, the technology of Patent Document 1 does not take into consideration the reduction in oxygen concentration in the room after carbon dioxide has been removed.

Therefore, an object of the present invention is to provide an air conditioning system and an air conditioning method that can separate and recover carbon dioxide in an air conditioning system in a building, and control the oxygen concentration in a room while effectively using the recovered carbon dioxide.

### Solution to Problem

The present invention has the following aspects to solve the problems described above.

[1] An air conditioning system includes a carbon dioxide separation device configured to separate some or all of carbon dioxide from air that contains carbon dioxide, an electrolytic reduction device configured to generate a hydrocarbon and oxygen using the separated carbon dioxide as a raw material, and an oxygen supply amount control device configured to supply some or all of the oxygen generated by the electrolytic reduction device to a space to be processed.

[2] The air conditioning system described in [1] further includes an ethylene glycol manufacturing device configured to generate ethylene glycol using some or all of the hydrocarbon generated by the electrolytic reduction device as a raw material.

The air conditioning system described in [2] in which the oxygen supply amount control device further includes a second oxygen flow rate regulator configured to supply some of the oxygen generated by the electrolytic reduction device to the ethylene glycol manufacturing device.

[4] The air conditioning system described in [2] or [3] further includes a hydrocarbon amount control device configured to adjust a flow rate of the hydrocarbon supplied to the ethylene glycol manufacturing device.

[5] The air conditioning system described in any one of [1] to [4] in which the air containing carbon dioxide is air discharged from the space to be processed.

The air conditioning system described in any one of [1] to [5] in which air inside the space to be processed is isolated from the outside.

[7] An air conditioning method includes a carbon dioxide separation process of separating some or all of carbon dioxide from air containing carbon dioxide; an electrolytic reduction process of generating a hydrocarbon and oxygen using the separated carbon dioxide as a raw material, and an oxygen supply amount control process of supplying some or all of the oxygen generated by the electrolytic reduction process to a space to be processed.

### Advantageous Effects of Invention

According to the air conditioning system and the air conditioning method of the present invention, in the air conditioning system for a building, it is possible to separate and recover carbon dioxide, and to control an oxygen concentration in a room while effectively using the recovered carbon dioxide.

### BRIEF DESCRIPTION OF DRAWINGS

FIG. 1 is a schematic diagram which shows an air conditioning system according to one embodiment of the present invention.

FIG. 2 is a schematic diagram which shows a flow of an air conditioning method according to one aspect of the present invention.

### DESCRIPTION OF EMBODIMENTS

### <<Air Conditioning System>>

The air conditioning system of the present invention includes a carbon dioxide separation device, an electrolytic reduction device, and an oxygen supply amount control device.

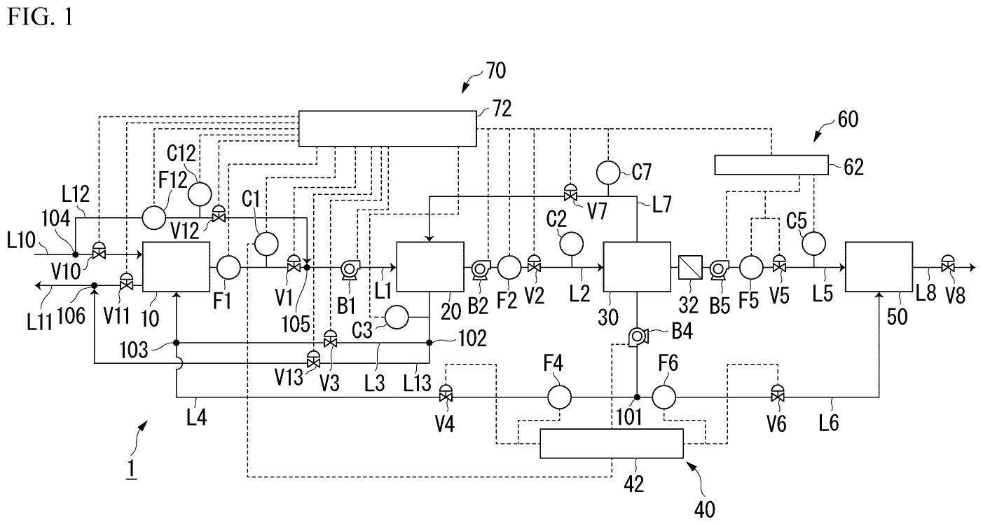

The air conditioning system according to a first embodiment of the present invention will be described in detail below with reference to FIG. 1.

As shown in FIG. 1, an air conditioning system 1 of the present embodiment includes a space to be processed 10, a carbon dioxide separation device 20, an electrolytic reduction device 30, an oxygen supply amount control device 40, an ethylene glycol manufacturing device 50. a hydrocarbon amount control device 60, and a carbon dioxide amount control device 70.

The space to be processed 10 and the carbon dioxide separation device 20 are connected by piping L1. The piping L1 is provided with a flow meter F1, a concentration meter C1, an opening and closing valve V1, and a blower B1. The carbon dioxide separation device 20 and the electrolytic reduction device 30 are connected by piping L2. The piping L2 is provided with a blower B2, a flow meter F2, an opening and closing valve V2, and a concentration meter C2. The electrolytic reduction device 30 and the ethylene glycol manufacturing device 50 are connected by piping L5. The piping L5 is provided with a hydrocarbon separation device 32, a blower B5, a flow meter F5, a hydrocarbon flow rate regulator V5, and a concentration meter C5.

The electrolytic reduction device 30 is connected to a piping LA, and the piping L4 is connected to the space to be processed 10. The piping LA is provided with a blower B4, a flow meter F4, and a first oxygen flow rate regulator V4. The carbon dioxide separation device 20 is connected to a piping L3, and the piping L3 is connected to the piping L4 via a branch 103. A branch 101 provided in the piping LA is connected to a piping L6, which is connected to the ethylene glycol manufacturing device 50. The piping L6 is provided with a flow meter F6 and a second oxygen flow rate regulator V6. The concentration meter C1, the first oxygen flow rate regulator V4, the flow meter F4, the blower B4, the flow meter F6, and the second oxygen flow rate regulator V6 are electrically connected to the controller 42, and constitute the oxygen supply amount control device 40. In the present specification, “electrically connected” means that transmission and reception of information can be performed, and it does not matter whether it is performed in a wired or wireless manner. The ethylene glycol manufacturing device 50 is connected to a piping L8. The piping L8 is provided with an opening and closing valve V8.

The electrolytic reduction device 30 is connected to a piping L7, which is connected to the carbon dioxide separation device 20. A concentration meter C7 and an opening and closing valve V7 are provided in the piping L7. The space to be processed 10 is connected to a piping L10 and a piping L11. An opening and closing valve V10 is provided in the piping L10. An opening and closing valve V11 is provided in a piping L11. A branch 104 provided in the piping L10 is connected to a piping L12, and the piping L12 is connected to the piping L1 at a branch 105. A flow meter F12, a concentration meter C12, and an opening and closing valve V12 are provided in the piping L12. A branch 102 provided in the piping L3 is connected to a piping L13, and the piping L13 is connected to the piping L11 at a branch 106. The piping L13 is provided with an opening and closing valve V13.

The blower B5, the flow meter F5, the hydrocarbon flow rate regulator V5, and the concentration meter C5 are electrically connected to the controller 62 and form the hydrocarbon amount control device 60. The flow meter F1, the concentration meter C1, the opening and closing valve V1, the blower B1, the blower B2, the flow meter F2, the opening and closing valve V2, the concentration meter C2, the opening and closing valve V3, the concentration meter C7, the opening and closing valve V7, the opening and closing valve V10, the opening and closing valve V11, the flow meter F12, the concentration meter C12, the opening and closing valve V12, the opening and closing valve V13, the oxygen supply amount control device 40, and the hydrocarbon amount control device 60 are electrically connected to the controller 72 to form the carbon dioxide amount control device 70.

An arrow in the figure indicates a direction of movement of a fluid such as air. Sensors (not shown) and the like are provided so that a temperature, a humidity, and a pressure of the fluid, which are necessary to derive an amount of substance of the fluid such as air, can be appropriately measured as necessary.

### <Space to be Processed>

The space to be processed 10 is an indoor space where an oxygen concentration is controlled. Examples of the space to be processed 10 include rooms where people are active indoors, such as offices. In the space to be processed 10, a concentration of carbon dioxide increases when people breathe. In the space to be processed 10, the concentration of oxygen decreases when people breathe.

In addition, when a flammable substance is burned using a heating appliance or the like in the space to be processed 10 to adjust a temperature of a room space, the concentration of carbon dioxide in the space to be processed 10 increases and the concentration of oxygen decreases.

### <Carbon Dioxide Separation Device>

The carbon dioxide separation device 20 is a device that separates some or all of the carbon dioxide from air that contains carbon dioxide. In the present specification, the carbon dioxide separation device 20 is defined as a device that does not discard the separated carbon dioxide, but recovers it and makes effective use of it.

Examples of the carbon dioxide separation device 20 include, but are not limited to, an adsorption or desorption device equipped with an adsorbent agent capable of adsorbing and desorbing carbon dioxide in air, an adsorption or desorption device equipped with an adsorbent agent capable of adsorbing and desorbing nitrogen in air, an adsorption or desorption device equipped with an adsorbent agent capable of adsorbing and desorbing oxygen in air, a membrane separation device equipped with a separation membrane capable of membrane separation of carbon dioxide, and the like.

Examples of the adsorbent agent capable of adsorbing and desorbing carbon dioxide in air include zeolite, silica gel, activated carbon, solid absorbent agents carrying amines such as triethanolamine and monoethanolamine, amine-based weakly basic anion exchange resins, and the like. Examples of adsorbent agents capable of adsorbing and desorbing carbon dioxide in air are preferably zeolite, silica gel, and activated carbon, and more preferably zeolite and silica gel.

Examples of the adsorbent agent capable of adsorbing and desorbing nitrogen in air include zeolite substituted with or carrying lithium, and the like.

Examples of the adsorbent agent capable of adsorbing and desorbing oxygen in air include perovskite-type adsorbents such as SrFeOx, BaFeOx, SrNiOx, and SrCoOx (x represents the number of oxygen atoms in each structure).

Examples of the separation membranes capable of membrane separation of carbon dioxide include carbon membranes with hollow-fiber-shaped porous carbon fibers as a support body.

When carbon dioxide is desorbed, any of a temperature swing adsorption (TSA) principle using a temperature difference, a pressure swing adsorption (PSA) principle using a pressure difference, and a vacuum swing adsorption (VSA) principle in which the pressure inside the carbon dioxide separation device 20 is reduced to 100 kPa or less to desorb carbon dioxide may be used.

The carbon dioxide separation device 20 of the present embodiment is a device that separates carbon dioxide from air discharged from the space to be processed 10.

In the space to be processed 10, since the concentration of carbon dioxide increases when a person breathes, efficiency of separating carbon dioxide can be further improved.

The carbon dioxide separation device 20 may be configured to separate carbon dioxide from air that flows through the piping L12 without passing through the space to Examples of the air that does not pass through the space to be processed 10 include air in a room other than the space to be processed 10, outdoor air (atmosphere), and the like.

be processed 10.

### <Electrolytic Reduction Device>

The electrolytic reduction device 30 is a device that generates a hydrocarbon and oxygen using carbon dioxide as a raw material.

Examples of the electrolytic reduction device 30 include an electrolysis device described in Japanese Unexamined Patent Application, First Publication No. 2018-150596, an electrolysis device described in Japanese Unexamined Patent Application, First Publication No. 2019-44238, and an electrolytic reduction device described in PCT International Publication No. WO 2022/049638, and the like, but are not particularly limited thereto.

### <Oxygen Supply Amount Control Device>

The oxygen supply amount control device 40 is a device that supplies some or all of the oxygen generated by the electrolytic reduction device 30 to the space to be processed 10.

The oxygen supply amount control device 40 of the present embodiment has a controller 42, a concentration meter C1, a blower B4, a flow meter F4, a first oxygen flow rate regulator V4, a flow meter F6, and a second oxygen flow rate regulator V6. The oxygen supply amount control device 40 uses the concentration meter C1 to monitor an oxygen concentration inside the space to be processed 10 and the flow meter F4 to monitor a flow rate of oxygen flowing through the piping L4, and supplies some or all of the oxygen generated by the electrolytic reduction device 30 to the space to be processed 10 while adjusting an opening of the first oxygen flow rate regulator V4 and an output value (power or frequency, or the like) of the blower B4.

The oxygen supply amount control device 40 monitors the flow rate of oxygen flowing through the piping L6 using the flow meter F6, and can supply some of the oxygen generated by the electrolytic reduction device 30 to the ethylene glycol manufacturing device 50 while adjusting the opening of the second oxygen flow rate regulator V6 and the output value (power, a frequency, or the like) of the blower B4.

### <Ethylene Glycol Manufacturing Device>

The ethylene glycol manufacturing device 50 is a device that generates ethylene glycol using some or all of the hydrocarbon generated by the electrolytic reduction device 30 as a raw material.

Examples of the ethylene glycol manufacturing device 50 include a reaction device described in Japanese Unexamined Patent Application, First Publication No. 2000-128814, Japanese Unexamined Patent Application, First Publication No. 2000-143562, and Japanese Unexamined Patent Application, First Publication No. 2001-316308, and the like, but are not limited thereto.

### <Hydrocarbon Amount Control Device>

The hydrocarbon amount control device 60 is a device which supplies some or all of the hydrocarbon generated by the electrolytic reduction device 30 to the ethylene glycol manufacturing device 50.

The hydrocarbon amount control device 60 in the present embodiment has the controller 62, the blower B5, the flow meter F5, the hydrocarbon flow rate regulator V5, and the concentration meter C5.

The hydrocarbon amount control device 60 monitors a flow rate of the hydrocarbon passing through the piping L5 using the flow meter F5, and supplies some or all of the hydrocarbon generated in the electrolytic reduction device 30 to the ethylene glycol manufacturing device 50 while adjusting an opening of the hydrocarbon flow rate regulator V5 and an output value (power, frequency, or the like) of the blower B5.

It is preferable that the hydrocarbon amount control device 60 sends a monitoring value monitored using the flow meter F5 to the carbon dioxide amount control device 70. The carbon dioxide amount control device 70 can adjust the opening degree of the opening and closing valve of each piping according to the monitoring value.

### <Carbon Dioxide Amount Control Device>

The carbon dioxide amount control device 70 is a device that controls an amount of carbon dioxide supplied from the carbon dioxide separation device 20 to the electrolytic reduction device 30. By having the carbon dioxide amount control device 70, the air conditioning system 1 can optimize the amount of carbon dioxide supplied from the carbon dioxide separation device 20 to the electrolytic reduction device 30. The carbon dioxide amount control device 70 of the present embodiment has a controller 72, a flow meter F1, a concentration meter C1, an opening and closing valve V1, a blower B1, a blower B2, a flow meter F2, an opening and closing valve V2, a concentration meter C2, an opening and closing valve V3, a concentration meter C7, an opening and closing valve V7, an opening and closing valve V10, an opening and closing valve V11, a flow meter F12, a concentration meter C12, an opening and closing valve V12, an opening and closing valve V13, an oxygen supply amount control device 40, and a hydrocarbon amount control device 60.

The carbon dioxide amount control device 70 adjusts the amount of carbon dioxide supplied from the carbon dioxide separation device 20 to the electrolytic reduction device 30 on the basis of a monitoring value monitored by the flow meter F5 and a hydrocarbon concentration measured by the concentration meter C5. More specifically, carbon dioxide is supplied to the electrolytic reduction device 30 while adjusting the opening of the opening and closing valve V2 and the output value (power, a frequency, or the like) of the blower B2 on the basis of the monitoring value described above.

A reaction amount of carbon dioxide in the electrolytic reduction device 30 is derived based on a supply amount of carbon dioxide supplied from the carbon dioxide separation device 20 to the electrolytic reduction device 30, a concentration of carbon dioxide flowing through the piping L7, a flow rate of hydrocarbon flowing through the piping L5, and a flow rate of oxygen flowing through the piping L4 and the piping L6.

In the carbon dioxide amount control device 70, an amount of carbon dioxide necessary to balance with the reaction amount of carbon dioxide in the electrolytic reduction device 30 is supplied while measuring flow rate values of the flow meters F1 and F12 and concentration values of the concentration meters C1, C3 and C12, and adjusting the opening of the opening and closing valves V1, V3, V10, V11, V12 and V13 and the output value (power, a frequency, or the like) of the blower B1.

Examples of the piping L1 include, for example, a duct made of metal or resin, and the like.

Examples of the pipings L2 to L7 include, for example, ducts similar to piping L1, and the like.

Examples of the piping L8 include, for example, a pipeline capable of transporting ethylene glycol, and the like.

Examples of the pipings L10 and L12 include, for example, ducts having an outside air intake port, and the like.

Examples of the pipings L11 and L13 include, for example, ducts having an exhaust port, and the like.

Examples of the opening and closing valve V1 include, for example, a solenoid valve whose opening and closing can be controlled by a computer or the like, and the like.

Examples of the opening and closing valves V2, V3, V7, V8, V10 to V13 include, for example, a solenoid valve similar to the opening and closing valve V1, and the like.

Examples of the first oxygen flow rate regulator V4, the second oxygen flow rate regulator V6, and the hydrocarbon flow rate regulator V5 include a solenoid valve similar to the opening and closing valve V1, and the like.

Examples of the blower B1 include a blower that provides energy to gas by a rotational motion of an impeller, and the like.

Examples of the blowers B2, B4, and B5 include blowers similar to the blower B1, and the like.

Examples of the flow meter F1 include a gas flow meter that can measure a flow rate of air, and the like.

Examples of the flow meters F2, F4 to F6, and F12 include a gas flow rate similar to the flow meter F1, and the like.

Examples of the concentration meter C1 include a concentration sensor that can measure an oxygen concentration, a carbon dioxide concentration, and a hydrocarbon concentration, and the like.

Examples of the concentration meters C2, C3, C5, C7, and C12 include a concentration sensor similar to the concentration meter C1, and the like.

Examples of the hydrocarbon separation device 32 include a distillation device capable of selectively separating a hydrocarbon, and the like.

Examples of the controller 42 include a computer capable of controlling an opening of the first oxygen flow rate regulator V4 and the second oxygen flow rate regulator V6, and an output value of the blower B4, and the like. Examples of the controller 62 include a computer capable of controlling an opening of the hydrocarbon flow rate regulator V5 and an output value of the blower B5, and the like.

Examples of the controller 72 include a computer capable of controlling an opening of the opening and closing valves V1, V3, V10, V11, V12, and V13, and an output values of the blowers B1 and B2, and the like.

### <<Air Conditioning Method>>

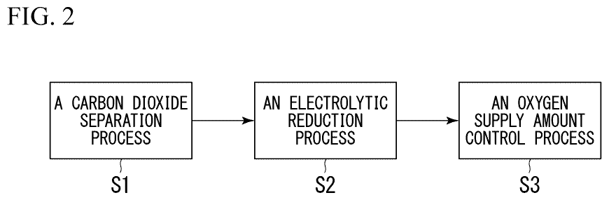

As shown in FIG. 2, the air conditioning method of the present invention includes a carbon dioxide separation process (S1), an electrolytic reduction process (S2), and an oxygen supply amount control process (S3).

The air conditioning method of the present embodiment will be described using an air conditioning method that uses an air conditioning system 1 as an example. Each process will be described in detail below with reference to FIG. 1.

First, the opening and closing valves V10 to V12 are closed, and the opening and closing valve V1 is opened.

By operating the blower B1, air inside the space to be processed 10 flows into the carbon dioxide separation device 20 through the piping L1 as air to be processed. By closing the opening and closing valve V1 and opening the opening and closing valve V12, atmosphere can also be supplied to the carbon dioxide separation device 20. For example, even when there is no one in the space to be processed 10, such as at night, it is possible to separate and recover carbon dioxide in the atmosphere by supplying the atmosphere to the carbon dioxide separation device 20.

The concentration of carbon dioxide in the air to be processed is, for example, preferably 100 to 5,000 ppm, more preferably 200 to 4,000 ppm, even more preferably 300 to 3,000 ppm, even more preferably 400 to 2,000 ppm, particularly preferably 500 to 1,500 ppm, and most preferably 600 to 1,000 ppm. When the concentration of carbon dioxide in the air to be processed is equal to or higher than the lower limit, a higher concentration of carbon dioxide can be supplied to the electrolytic reduction device 30. When the concentration of carbon dioxide in the air to be processed is equal to or less than the upper limit, the concentration of carbon dioxide contained in the air after separation of carbon dioxide (processed air) can be further reduced.

### <Carbon Dioxide Separation Process>

The carbon dioxide separation process (S1) is a process for separating some or all of carbon dioxide from air containing carbon dioxide (air to be processed). In the present embodiment, the air to be processed is supplied to the carbon dioxide separation device 20, and carbon dioxide is separated by the carbon dioxide separation device 20.

When the carbon dioxide separation device 20 is an adsorption or desorption device, the air to be processed is brought into contact with an adsorbent agent, and any one of carbon dioxide, nitrogen, and oxygen in the air to be processed is adsorbed by the adsorbent agent.

When carbon dioxide is adsorbed in this process, the processed air from which some or all of the carbon dioxide has been removed is caused to pass through the piping L3, the branch 103, and the piping L4 and is supplied to the space to be processed 10 with the opening and closing valve V3 opened. By desorbing carbon dioxide from the adsorbent agent to which carbon dioxide has been adsorbed, the desorbed carbon dioxide (carbon dioxide-rich gas) can be supplied to the electrolytic reduction device 30.

When nitrogen is adsorbed in this process, some or all of the nitrogen is removed, and a gas with a relatively high oxygen concentration (oxygen-rich gas) is obtained. The oxygen-rich gas is further brought into contact with an adsorbent agent to cause the adsorbent agent to adsorb some or all of the oxygen. The gas from which some or all of the oxygen has been removed and has a relatively high carbon dioxide concentration (carbon dioxide-rich gas) is supplied to the electrolytic reduction device 30.

Nitrogen-rich gas is obtained by desorbing the nitrogen adsorbed by the adsorbent agent. The nitrogen-rich gas may be recovered as nitrogen, or may be discharged to the outdoors by closing the opening and closing valve V3, opening the opening and closing valve V13, and allowing the gas to flow through the piping L3, the branch 102, the piping L13, the branch 106, and the piping L11.

Oxygen-rich gas is obtained by desorbing the oxygen adsorbed by the adsorbent agent. The oxygen-rich gas may be supplied to the space to be processed 10 by opening the opening and closing valve V3, and allowing the gas to flow through the piping L3, the branch 103, and the piping L4, or may be supplied to the ethylene glycol manufacturing device 50.

Nitrogen-rich gas and oxygen-rich gas may be mixed and supplied to the space to be processed 10 as processed air.

When the carbon dioxide separation device 20 is a membrane separation device, the air to be processed is caused to pass through the separation membrane to separate some or all of the carbon dioxide being processed. The separated carbon dioxide (carbon dioxide-rich gas) is supplied to the electrolytic reduction device 30. The processed air from which some or all of the carbon dioxide has been removed is supplied to the space to be processed 10.

For example, when the atmosphere (outside air) is directly supplied to the carbon dioxide separation device 20 by bypassing the space to be processed 10 at night, or the like, and passing through the piping L12, the processed air may be discharged to the outdoors by opening the opening and closing valve V13 and passing through the piping L3, the branch 102, the piping L13, the branch 106, and the piping L11.

The separated carbon dioxide (carbon dioxide-rich gas) flows through the piping L2 and flows into the electrolytic reduction device 30 by opening the opening and closing valve V2 and operating the blower B2.

The concentration of carbon dioxide in the carbon dioxide-rich gas may be, for example, 1,000 ppm or more, preferably 1,000 to 750,000 ppm, 1,000 to 500,000 ppm, 1,000 to 250,000 ppm, or 1,000 to 100,000 ppm, more preferably 1,000 to 10,000 ppm, even more preferably 2,000 to 10,000 ppm, and particularly preferably 2,000 to 5,000 ppm. When the concentration of carbon dioxide in the carbon dioxide-rich gas is equal to or higher than the lower limit, a larger amount of carbon dioxide can be supplied to the electrolytic reduction device 30. When the concentration of carbon dioxide in the carbon dioxide-rich gas is equal to or lower than the upper limit, management becomes easier.

The concentration of carbon dioxide in the carbon dioxide-rich gas can be adjusted by a type and an amount of an adsorbent agent, a type and a shape of a separation membrane, an internal pressure of the carbon dioxide separation device 20, an internal temperature of the carbon dioxide separation device 20, or a combination of these.

The concentration of carbon dioxide in the carbon dioxide-rich gas can be measured by, for example, the concentration meter C2.

The processed air from which carbon dioxide has been separated flows through the piping L3 and flows into the space to be processed 10 by opening the opening and closing valve V3.

The carbon dioxide concentration in the processed air is preferably 1,000 ppm or less, more preferably 800 ppm or less, and even more preferably 500 ppm or less. When the carbon dioxide concentration in the processed air is equal to or less than the upper limit, the carbon dioxide concentration can be made to satisfy the building environmental sanitation management standards, and cleaner air can be supplied to the space to be processed 10. A lower limit of the carbon dioxide concentration in the processed air is not particularly limited, but is substantially 10 ppm, and may be 0 ppm.

### <Electrolytic Reduction Process>

The electrolytic reduction process (S2) is a process for generating a hydrocarbon and oxygen using the separated carbon dioxide as a raw material. In the present embodiment, the carbon dioxide separated in the carbon dioxide separation process is supplied to the electrolytic reduction device 30 by which electrolytic reduction processing is performed.

Examples of the generated hydrocarbon include methane, ethane, and ethylene. Methane and ethylene are preferred hydrocarbons because they are the raw materials for ethylene glycol, and ethylene is more preferred.

In the electrolytic reduction process, it is preferable to perform an enrichment operation to increase the concentration of the separated carbon dioxide. Examples of the enrichment operation include a method of performing electrochemical processing.

By including an enrichment operation in the electrolytic reduction process, efficiency of a reaction to obtain hydrocarbon, which will be described below, can be further increased.

In the electrolytic reduction process, for example, carbon dioxide is reduced to obtain hydrocarbon by supplying carbon dioxide and water to a cathode electrode. A gas diffusion electrode or the like is preferred as a structure of the cathode electrode. In this case, oxygen is generated from the cathode electrode as a by-product. The generated oxygen flows into the piping L4.

The obtained hydrocarbon passes through the hydrocarbon separation device 32, and flows through the piping L5 and flows into an ethylene glycol manufacturing device 50 by opening the hydrocarbon flow rate regulator V5 and operating the blower B5.

### <Oxygen Supply Amount Control Process>

The oxygen supply amount control process (S3) is a process in which some or all of the oxygen obtained in the electrolytic reduction process is supplied to the space to be processed 10. In the present embodiment, the flow rate of oxygen supplied to the space to be processed 10 is controlled by the oxygen supply amount control device 40 monitoring the flow rate of oxygen using the flow meter F4, and adjusting the opening of the first oxygen flow rate regulator V4 and the output value (power, a frequency, or the like) of the blower B4. The oxygen that flows into the piping L4 is supplied to the space to be processed 10 while its flow rate is controlled by the oxygen supply amount control device 40.

In the space to be processed 10, the concentration of oxygen decreases when a person breathes. By supplying oxygen to the space to be processed 10 using the oxygen supply amount control process, the oxygen concentration in the space to be processed 10 is kept constant.

In consideration of effects on a human body, for example, the oxygen concentration in the space to be processed 10 is preferably 19% by volume or more, more preferably 20% by volume or more, and even more preferably 21% by volume or more. The oxygen concentration in the space to be processed 10 can be measured using, for example, an oxygen concentration meter (not shown) or the like provided in the space to be processed 10. The oxygen concentration in the space to be processed 10 may be monitored using an oxygen supply amount control device 40 or a carbon dioxide amount control device 70.

By monitoring the oxygen concentration in the space to be processed 10 using the oxygen supply amount control device 40 or the like, an amount of oxygen supplied to the space to be processed 10 and an amount of processed air supplied to the space to be processed 10 can be adjusted.

Some of the oxygen obtained in the electrolytic reduction process may be supplied to the ethylene glycol manufacturing device 50 through the piping L6 by closing the first oxygen flow rate regulator V4, opening the second oxygen flow rate regulator V6, and operating the blower B4.

A flow rate of the oxygen supplied to the ethylene glycol manufacturing device 50 can be controlled by the oxygen supply amount control device 40 by monitoring it using the flow meter F6 and adjusting an opening of the second oxygen flow rate regulator V6 and an output value (power, a frequency, or the like) of the blower B4. By supplying oxygen to the ethylene glycol manufacturing device 50, ethylene oxide, which is a raw material of ethylene glycol, can be generated.

### <Ethylene Glycol Manufacturing Process>

The ethylene glycol manufacturing process is a process of generating ethylene glycol using some or all of the hydrocarbon generated in the electrolytic reduction process as raw materials. In the present embodiment, some or all of the hydrocarbon obtained in the electrolytic reduction process are supplied to the ethylene glycol manufacturing device 50, and ethylene glycol is generated in the ethylene glycol manufacturing device 50.

In the ethylene glycol manufacturing process, first, the hydrocarbons are purified to obtain ethylene. Next, ethylene and oxygen are reacted in a presence of a silver catalyst in which silver is carried in an alumina carrier to obtain ethylene oxide.

The oxygen reacted with ethylene may be oxygen obtained in the electrolytic reduction process or oxygen supplied from the outside. From a viewpoint of increasing energy efficiency, oxygen obtained in the electrolytic reduction process is preferable as the oxygen reacted with ethylene.

In addition, methane may be added as a diluent to suppress an oxidation reaction of ethylene. The methane added as a diluent may be supplied from the outside or may be methane obtained in the electrolytic reduction process. From a viewpoint of increasing an energy efficiency, methane obtained in the electrolytic reduction process is preferable as methane added as a diluent.

A method for obtaining ethylene glycol from the obtained ethylene oxide is not particularly limited, and known methods can be used. Examples of the method for obtaining ethylene glycol include a method using a liquid phase hydration reaction without a catalyst, and a method of producing ethylene carbonate using a catalyst and then obtaining ethylene glycol.

Examples of catalysts include alkali metal iodides or bromides, alkaline earth metal halides, halogenated organic phosphonium salts, alkali metal carbonates, combinations of these, or the like.

The obtained ethylene glycol is purified, and then transferred to the outside through piping L8 by opening the opening and closing valve V8.

Ethylene glycol is safer than ethylene and is suitable for storage and preservation.

Ethylene glycol is useful as a raw material for polyethylene terephthalate (PET) and polyethylene naphthalate (PEN).

By opening the opening and closing valve V10, air from the outside may be passed through the piping L10 and supplied to the space to be processed 10. By opening the opening and closing valve V11, the air in the space to be processed 10 may be passed through the piping L11 and discharged to the outside. However, since this makes a significant reduction in energy possible, it is preferable to close the opening and closing valves V10 and V11 and to isolate the air inside the space to be processed 10 from the outside.

Here, “isolated from the outside” means that the space to be processed 10 does not have an exchange port with the outside air. In the present embodiment, the piping L10 and the piping L11 connected to the space to be processed 10 are the exchange ports with the outside air. However, by closing both opening and closing valves V10 and V11, the air inside the space to be processed 10 is isolated from the outside, resulting in a state in which there is “no exchange port with the outside air.”

By applying the air conditioning system of the present embodiment to a space to be processed that does not have an exchange port with the outside air, it is possible to maintain a constant oxygen concentration in the space even in a space that is closed off from the outside, such as a space station, a submarine, or a nuclear shelter.

As described above, the air conditioning system of the present embodiment can separate carbon dioxide from the outside air and air inside the space to be processed. For this reason, air with a reduced carbon dioxide concentration can be supplied to the space to be processed.

The air conditioning system of the present embodiment can generate hydrocarbon from the separated carbon dioxide. For this reason, the separated carbon dioxide can be effectively used as an energy source such as a carbon source or as a raw material for basic chemicals.

The air conditioning system of the present embodiment can supply oxygen generated by the electrolytic reduction device to the space to be processed, so that the oxygen concentration in the space to be processed can be controlled. For this reason, it is not necessary to rely on the outside air for the air to be supplied to the space to be processed. As a result, it is possible to reduce an outside air load, which is said to account for 40% of an air conditioning load.

The air conditioning system of the present embodiment can reduce the air conditioning load, so that it is possible to reduce air conditioning costs and an energy required for air conditioning. This leads to a reduction in discharge amount of carbon dioxide from power plants.

The air conditioning system of the present embodiment can separate and directly recover carbon dioxide from the outside air, so if it is widely used, it will lead to a reduction in carbon dioxide across the globe. In addition, since it can separate and directly recover carbon dioxide from the outside air, it can recover carbon dioxide in large quantities and stably compared to a conventional technology in which carbon dioxide is absorbed only from an indoor exhaust.

The carbon dioxide separated by the air conditioning system or air conditioning method of the present embodiment can be stably supplied in an amount required for industrial use. For this reason, the separated carbon dioxide is suitable as a material for a synthesis of C1 compounds such as carbon monoxide, methane, methanol, and formic acid, a material for a synthesis of C2 compounds such as ethane, ethylene, and ethanol, or a material for a synthesis of olefin compounds such as propylene and butene.

The air conditioning system of the present embodiment makes it possible to obtain safe and highly useful ethylene glycol from the generated hydrocarbon. In this manner, the technology of the present invention is a technology that is beneficial to the global environment.

Although the preferred embodiment of the present invention has been described in detail, but the present invention is not limited to such a specific embodiment, and various modifications are possible within the scope of the gist of the present invention described in the claims.

In the embodiment described above, the air discharged from the space to be processed is set to air to be processed, but the outdoor air may also be set to the air to be processed. By setting the outdoor air to the air to be processed, carbon dioxide can be continuously supplied to the electrolytic reduction device.

By setting the outdoor air to the air to be processed, the carbon dioxide concentration in the atmosphere can be reduced.

In the embodiment described above, there is one space to be processed, but there may be two or more spaces to be processed. By increasing the number of spaces to be processed, it becomes possible to separate and recover more carbon dioxide. As a result, a production volume of the obtained hydrocarbon can be further increased.

For example, the air conditioning system of the present invention can be applied to structures such as buildings that have spaces to be processed on two or more floors. By applying the air conditioning system of the present invention to a plurality of buildings, it becomes possible to realize a district heating and cooling system supply block.

The number and installation positions of pipings, flow meters, opening and closing valves, concentration meters, blowers, and the like are not limited to those of the embodiment described above, and any number of these can be installed in any positions within the scope of the gist of the present invention.

The “Sustainable Development Goals (SDGs)” are 17 international goals that have been adopted at the United Nations Summit in September 2015. An air conditioning system according to one embodiment can contribute to an achievement of a goal, for example, “goal 7: Affordable and clean energy” or the like among the 17 SDGs.

### REFERENCE SIGNS LIST

1 Air conditioning system

10 Space to be processed

20 Carbon dioxide separation device

30 Electrolytic reduction device

32 Hydrocarbon separation device

40 Oxygen supply amount control device

42, 62, 72 Controller

50 Ethylene glycol manufacturing device

60 Hydrocarbon amount control device

70 Carbon dioxide amount control device

V1, V2, V3, V7, V8, V10, V11, V12, V13 Opening and closing valve

V4 First oxygen flow rate regulator

V5 Hydrocarbon flow rate regulator

V6 Second oxygen flow rate regulator

L1 to L8, L10 to L13 Piping

F1, F2, F4 to F6, F12 Flow meter

C1, C2, C3, C5, C7, C12 Concentration meter

B1, B2, B4, B5 Blower

101, 102, 103, 104, 105, 106 Branch

1. An air conditioning system comprising:
a carbon dioxide separation device configured to separate some or all of carbon dioxide from air that contains carbon dioxide;
an electrolytic reduction device configured to generate a hydrocarbon and oxygen using the separated carbon dioxide as a raw material; and
an oxygen supply amount control device configured to supply some or all of the oxygen generated by the electrolytic reduction device to a space to be processed.

2. The air conditioning system according to claim 1, further comprising an ethylene glycol manufacturing device configured to generate ethylene glycol using some or all of the hydrocarbon generated by the electrolytic reduction device as a raw material.

3. The air conditioning system according to claim 2, wherein the oxygen supply amount control device further includes a second oxygen flow rate regulator configured to supply some of the oxygen generated by the electrolytic reduction device to the ethylene glycol manufacturing device.

4. The air conditioning system according to claim 2, further comprising a hydrocarbon amount control device configured to adjust a flow rate of the hydrocarbon supplied to the ethylene glycol manufacturing device.

5. The air conditioning system according to claim 1, wherein the air containing carbon dioxide is air discharged from the space to be processed.

6. The air conditioning system according to claim 1, wherein air inside the space to be processed is isolated from the outside.

7. An air conditioning method comprising:
a carbon dioxide separation process of separating some or all of carbon dioxide from air containing carbon dioxide;
an electrolytic reduction process of generating a hydrocarbon and oxygen using the separated carbon dioxide as a raw material; and
an oxygen supply amount control process of supplying some or all of the oxygen generated by the electrolytic reduction process to a space to be processed.
